(19) United States
(12) Patent Application Publication
(10) Pub. No.: US 2010/0268012 A1
(43) Pub. Date: Oct. 21, 2010

(54) Radioprotective materials, methods of transport and utilization thereof, nanoscale-microscale supramagnetic and supraconducting particles, spherical flow dynamics and sonoluminesence

(76) Inventors: Patrick Allen Plummer, Scottsdale, AZ (US); Marina Lisa Zakala, Surprise, AZ (US); Mack Carter Plummer, Paradise Valley, AZ (US)

Correspondence Address: Marina Lisa Zakala, 11673 West Hedgehog Ct, Surprise, AZ, 85374, US

(21) Appl. No.: 11/655,342

(22) Filed: Jan. 19, 2007

(51) Int. Cl. A61N 5/00; G05D 7/00

(52) U.S. Cl. 600/1; 137/1

(57) ABSTRACT

A spherical sonoluminesence chamber is treated with a positively charged transducer at one end and a negatively charged transducer at the other end, and a charge is applied to a medium of helium or other gas or liquid, with the anions, cat ions, or oxides of our selected particles dispersed therein. With the application of sufficient energy, a supraconducting entity is thus achieved via cooper-pair bonding, with techniques for enhancing the entity's paraconducting and supraconducting properties. Applications for using these novel entities in a variety of arenas are disclosed.

This application claims priority to U.S. Provisional Application No. 164,185 filed on Jan. 20, 2006 by the same parties.

### BACKGROUND OF THE INVENTION

The earliest reference to a sonofusion-type reaction is in U.S. Pat. No. 4,333,796 filed by Hugh Flynn in 1978. Rusi P. Taleyarkhan (ORNL) and colleagues reported in the Mar. 8, 2002, issue of the peer-reviewed journal Science, that acoustic cavitation experiments conducted with deuterated acetone (C₃D₆O) show measurements of tritium and neutron output that are consistent with fusion; in addition the neutron emission was claimed to be coincident with the sonoluminescence pulse.^{[1]}

Shock wave simulations seem to indicate that the temperatures inside the collapsing bubbles may reach up to 10 megakelvins—as hot as the center of the sun. None of the above measurements have been confirmed by a group outside of Taleyarkhan's and are highly debated, recalling the 1989 cold fusion fiasco.^{[2] [3] [4] [5]} However, New Energy Times has reported a replication by an unrelated group at the University of Texas.^{[6]} Although the apparatus operates in a room temperature environment, this is not cold fusion (as it is commonly termed in the popular press), as the claimed nuclear reactions would be occurring at the very high temperatures in the core of the imploding bubbles.

The researchers used a pulse of neutrons in order to nucleate (i.e., “seed”) the tiny bubbles, whereas most previous experiments start with small air bubbles already in the liquid. Using this new method, the team was able to produce stable bubbles that could expand to nearly a millimeter in radius before collapsing. In this way, the researchers stated, they were able to create the conditions necessary to produce very high pressures and temperatures. The sensitivity of the fusion rate to temperature, which is in turn a function of how small the bubbles get when they collapse, in combination with the likely sensitivity of the latter to fine experimental details, may account for the fact that some research workers have claimed to see an effect, while others have not.

Taleyarkhan et al. also prepared identical experiments in non-deuterated (normal) acetone and failed to observe neutron emission or tritium production. Taleyarkhan got the idea of bubble fusion from his friend Dr. Mark Embrechts after a friendly post-dinner chat in 1995.

### Oak Ridge Replication

These experiments were repeated at Oak Ridge National Laboratory by D. Shapira and M. J. Saltmarsh with more sophisticated neutron detection equipment and they reported that the neutron release was consistent with random coincidence.^{[7]} A rebuttal by Taleyarkhan and the other authors of the original report claimed that the Shapira and Saltmarsh report failed to account for significant differences in experimental setup, including over an inch of shielding between the neutron detector and the sonoluminescing acetone. Taleyarkhan et al. report that when these differences are properly accounted for, the Shapira and Saltmarsh results are consistent with fusion.

In addition, Galonsky has shown that by Taleyarkhan's own detector calibration the observed neutrons are too high in energy to be from a d-d fusion reaction. In a rebuttal comment, Taleyarkhan says the energy is “reasonably close” to that which is expected.^{[8]}

In February 2005, the BBC Horizon documentary commissioned a collaboration between Seth Putterman and Ken Suslick (two leading sonoluminescence researchers) to reproduce Taleyarkhan's work. Using similar acoustic parameters, deuterated acetone, similar bubble nucleation, and a much more sophisticated neutron detection device, the researchers could find no evidence of a fusion reaction. This work was reviewed by a team of four scientists, including an expert in sonoluminescence and an expert in neutron detection, who also concluded that no evidence of fusion could be observed.^{[9]}

### Subsequent Claims of Replication

In 2004, new claims of bubble fusion were made by the Taleyarkhan group, claiming that the results of previous experiments have been replicated under more stringent experimental conditions.^{[10][11]} These results differed from the original results in that fusion was occurring for a much longer time frame than previously reported; the original report only showed neutron emission from the initial bubble collapse after the bubble nucleation whereas this report shows neutron emission many acoustic cycles later. The data however was lacking in that too large of a window was used for determination of a coincidence between the neutron emission and sonoluminescence light emission. Also, the energy of the detected neutrons were not consistent with neutrons produced from a fusion reaction.

In July 2005, two of Taleyarkhan's students at Purdue University published evidence confirming the previous result.^{[12] [13]} They used the same acoustic chamber, the same deuterated acetone fluid and a similar bubble nucleation system. In this report, no neutron-sonoluminescence coincidence was attempted; also the neutron energy was again not consistent with a neutron produced from a d-d fusion reaction.

A report published in the journal Physical Review Letters claims further evidence of fusion.^{[14] [15] [16] [17]} The initial news report, however, shows that the reaction does not always work correctly, and it is not known what parameters change to cause the reaction to function properly versus not function at all.

### Doubts Prompt Investigation

A claim as spectacular as that one naturally arouses a lot of doubt. This culminated in a “special report” published in March 2006 by Nature, that seriously calls into question the validity of the results of the Purdue experiments.^{[18]} They quote Brian Naranjo of the University of California, Los Angeles with the claim^{[19]} that the measured spectrum is consistent with radioactive decay of the lab equipment and hence does not necessarily prove the presence of nuclear reactions (however, the response of Taleyarkhan et al. published in Physical Review Letters^{[20]} attempts to refute Naranjo's hypothesis as to the cause of the neutrons detected; the refutation, however, is itself flawed, and thereby adds no credibility to the original claim).

Doubts about the truthfulness of claims of positive observations have arisen within the Nuclear Engineering faculty of Purdue University. Because of extremely serious concerns, Purdue has initiated a review of the research, to be conducted by Purdue's Office of the Vice President for Research. In a March 9 article headed “Evidence for bubble fusion called into question”, Nature journal reported that it had interviewed several of Taleyarkhan's colleagues who suspect something is amiss.^{[21]}

### Nature's Suggestion of Misuse of Funds Ill-Founded?

In July 2006, Nature publicized a claim of Seth Putterman, denied by Taleyarkhan, to the effect that DARPA funds were used to support an experiment reported in Physical Review Letters without the source being acknowledged. This may seem a matter of minor importance. Questions as to Nature's motives^{[22]} have been raised by the fact that, in the article, a conspicuous display of Putterman's arguments headed ‘where did the money go?’ is immediately followed by a paragraph devoted to ‘misuse of federal dollars’. Since Putterman does not himself consider funds were misused, it is unclear why such a paragraph should have been included if there were no intent at all to make the reader think this might have been the case.

Another problem with Nature's stance (the journal has stated “[we] believe that we have nothing to apologise for, and nothing to correct”) is that the accounting details obtained by Putterman do not appear necessarily to support his conclusions. Despite the damaging effects that the publication of a flawed allegation is likely to have had, the journal is still at this time refusing to publish a clarification.^{[22]}

### Ethical Dimension

The ethical aspect of Nature's coverage of bubble fusion is complementary to that addressed in the above, and has equally been a cause of concern. Taking the Code of Ethics of the Society of Professional Journalists^{[23]} as the norm, one may note the following in relation to the article concerned:
1. According to the code, journalists should ‘test the accuracy of information from all sources and exercise care to avoid inadvertent error’. As noted above, the accounting details listed in the article do not appear to support Putterman's possibly damaging conclusions: however, they are presented in the article in a way that suggests that they do.
2. Journalists should also ‘make certain that headlines . . . do not misrepresent. They should not oversimplify or highlight incidents out of context.’ Merely posing the question ‘Where did the money go?’, which appears very conspicuously in the article, raises in the reader's mind the idea of fraud, a suggestion that the article itself does not in any way support.
   See also Science, Journalism and Nature.^{[24]}

### November 2006 Replication Claims

In November 2006, Edward R. “Ted” Forringer, Ph.D. and undergraduates David Robbins and Jonathan Martin of LeTourneau University presented two papers at the American Nuclear Society Winter Meeting, claiming replication of neutron emission during a visit to the meta-stable fluids research lab at Purdue University. Their experimental setup was similar to the others, using a mixture of deuterated acetone, deuterated benzine, tetrachloroethylene and uranyl nitrate and, notably, operating without an external neutron source and using two types of neutron detectors. They measured neutron levels at 8 standard deviations above the background level with a liquid scintillation detector, and 3.8 standard deviations above the background with plastic detectors. Measurements were within one standard deviation for the same experiment with a non-deuterated control liquid, demonstrating neutron production only during cavitation of the deuterated liquid.^{[26][26][27]}

### See Also

Sonoluminescence—the emission of short bursts of light from imploding bubbles in a liquid when excited by sound.
Cold Fusion—research continues into cold fusion into the 21st Century.
List of energy topics
Chain Reaction the fictionalized tale of a discovery and attempted cover-up of a practical fusion reactor.

Sonoluminescence is a well-known phenomena discovered in the 1930's in which light is generated when a liquid is cavitated. Although a variety of techniques for cavitating the liquid are known (e.g., spark discharge, laser pulse, flowing the liquid through a Venturi tube), one of the most common techniques is through the application of high intensity sound waves.

In the past, a particular ultrasound method has been to treat a fountain solution including simultaneously exposing the fountain solution to gas microbubbles and high-frequency ultrasound. A particular device has also been provided, including a compartment for holding a reservoir of fountain solution, a gas microbubble emitter configured to emit microbubbles into the compartment, and a high-frequency ultrasound-emitter configured to emit ultrasound signals into the compartment. The high-frequency ultrasound is higher than 100 kHz.

The cavitation process consists of three stages; bubble formation, growth, and subsequent collapse. The bubble or bubbles cavitated during this process absorb the applied energy, for example sound energy, and then release the energy in the form of light emission during an extremely brief period of time. The intensity of the generated light depends on a variety of factors including the physical properties of the liquid (e.g., density, surface tension, vapor pressure, chemical structure, temperature, hydrostatic pressure, etc) and the applied energy (e.g., sound wave amplitude, sound wave frequency, etc.).

Although it is generally recognized that during the collapse of a cavitating bubble extremely high temperature plasmas are developed, leading to the observed sonoluminescence effect, we contend that most important aspects of the phenomena have until now not been characterized. As such, the phenomena is at the heart of a considerable amount of research as scientists attempt to further understand the phenomena (e.g., effects of varying degrees of pressure on the cavitating medium) as well as its many applications which may utilize one or more of the products (e.g., sonochemistry, chemical detoxification, ultrasonic cleaning, etc.).

U.S. Pat. No. 4,333,796 discloses a cavitation chamber that is generally cylindrical although the inventors note that other shapes, such as spherical, can also be used. It is further disclosed that the chamber is comprised of one of a limited number of refractory metals such as tungsten, titanium, molybdenum, rhenium or some alloy thereof and the cavitation medium is a liquid metal such as lithium or an alloy thereof. Surrounding the cavitation chamber is a housing which is purportedly used as a neutron and tritium shield. Projecting through both the outer housing and the cavitation chamber walls are a number of acoustic horns, each of the acoustic horns being coupled to a transducer which supplies the mechanical energy to the associated horn.

Similarly, U.S. Pat. No. 5,659,173 discloses a sonoluminescence system that uses a transparent spherical flask. The spherical flask is not described in detail, although the specification discloses that flasks of Pyrex®, Kontes®, and glass were used with sizes ranging from 10 milliliters to 5 liters. The drivers as well as a microphone piezoelectric were epoxied to the exterior surface of the chamber.

U.S. Pat. No. 6,361,747 discloses an acoustic cavitation reactor comprised of a flexible tube through which the liquid to be treated circulates. Electroacoustic transducers are radially and uniformly distributed around the tube, each of the electroacoustic transducers having a prismatic bar shape. As disclosed, the reactor tube may be comprised of a non-resonant material such as a resistant polymeric material (e.g., TFE, PTFE), with or without reinforcement (e.g., fiberglass, graphite fibers, mica).

PCT Application No. US02/16761 actually discloses a nuclear fusion reactor in which at least a portion of the liquid within the reactor is placed into a state of tension, this state of tension being less than the cavitation threshold of the liquid. In at least one disclosed embodiment, acoustic waves are used to pretension the liquid. After the desired state of tension is obtained, a cavitation initiation source, such as a neutron source, nucleates at least one bubble within the liquid, the bubble having a radius greater than a critical bubble radius. The nucleated bubbles are then imploded, the temperature generated by the implosion being sufficient to induce a nuclear fusion reaction.

Recently, modeling based on analogous phenomena and/or laboratory testing equipment to analogize the gas and fluid dynamics of the wave form can be used here to support the intensity of the sonic wave, pressure at various points in the vessel from the transducer to the nucleation area (reaction area).

In one example, the bubble temperature can increase to be over 10,000 K, though the effect of shock wave is neglected, partly due to the small molar heat of argon compared with air. The black-body radiation is examined as a possible mechanism of SBS L. See Kyuichi Yasui, Abstract New Model of Single-Bubble Sonoluminescence Department of Physics, Waseda University, 3-4-1 Ohkubo, Shimjuku, Tokyo, Japan

For example this is in the temperature range measured for the creation of new blue giant stars see below: The blue color of the sky is not due to the black body radiation, but rather to Rayleigh scattering of the sunlight from the atmosphere which tends to scatter blue light more than red light. This phenomenon has nothing to do with properties of a black body.

[Note this diagram is only a symbolic-representation; the colors shown have not been calculated with any colorimetric accuracy. A colorimetrically-accurate diagram is available here.

Some common examples.
1700 K: Light of matches
1850K: a candle
2800 K: tungsten lamp (ordinary household bulb whatever its power)**
3350K: studio “CP” light
3400 K: studio lamps, photofloods,
5000 K: Daylight°
5500 K: average daylight, electronic flash (can vary between manufacturers)
5770 K: effective sun temperature
6420 K: Xenon arc lamp
6500 K: Daylight°
9300 K: TV screen (analog)
28000-30000 K: a lightning bolt ild see SEE
http://en.wikipedia.org/wiki/Color_temperature

In sonic cavitation, the vortexes collapse (see Crandal and Plummer et.al, Star Generating System SGS, Life Extention Publications LLC 1998 herein after referred to as SGS).A provisional patent application was filed regarding the potential energy-generating aspects of a spherical dewar limited to helium gas in a frozen environmemt.

This same phenomena of the bubble collapsing is referred to as microjets see Suslick K., Sonolumnescence and sonochemistry, Encyclopedia of Science and Technology 3^{rd} Edition, R.A. Myers (ed), Academic Press Inc San Diego 2001 pg 5 hereafter Suslick K 2001

http://www.scs.uiuc.ed u/suslick/pdf/ap.encyclphysscitech2001.pdf

Also, extreme temperatures in the reaction area (nucleus area) can reach 5000 K and 1000 atmospheres of pressure (see Suslick K 2001 on pg 2-3). Ultrasound frequencies span 15 Khz thorough 1 Ghz are still larger than molecules and direct coupling has not been achieved (Suslick K 2001 on pg 3) Wavelengths of 10 to 10⁴ have been documented (Suslick K 2001 on pg 3). Very limited and solely liquid metal powder suspension has been experimented with (Suslick K 2001 on pg 3). The flash of SL is emitted at the minimum radius where the acceleration exceeds 10̂11 g. The upper limit of energy focusing can be achieved with SL has not yet been determined (see Seth Putterman, Abstract Defining the Unknowns of Sonoluminescence Department of Physics, UCLA, Los Angeles, Calif. 90095)

These speeds are consistent with ultra relativistic speeds, and consistent with those measured with radiotelescopes measuring vortex jets in galaxies where suns are suspected to be made (see SGS, also white dwarf discussion, pg 64, 65).

One hypothesis is that just before speeds are achieved reaching fusion that the coupling at the apex can occur consistent with the massubauer affect of resonant frequency wherby the radius of the tip of the apex vortex is the same as that of the atom or mano particle to grab hold of it in a gear like fashion and efficiently compress the atom or molecules atoms or sub atomic particles closer together resulting in a denser molecule or atom, yet one with a stronger magnetic field from the conserved energy.

Modern transducers and accompanying software are capable of resonating at speeds and sizes to effectuate coupling see US Patent # 20060272419 by Maris; Humphrey J; et al, Opto-acoustic methods and apparatus for performing high resolution acoustic imaging and other sample probing and modification operations headnote 50, 52,71,72, 130 and 73. see also, see US Patent # 20060223185 by Fedorov; Andreig; et al, ELECTROSONIC CELL MANIPULATION DEVICE AND METHOD OF USE THEREOF headnote 554-557 and as to health promoting biological effects of magnetized particles see headnote 1255,1260 see also US Patent # 20070001267 by Ayazi; Farrokh; et al, methods of forming oxide masks with submicron openings and microstructures formed thereby headnote 2-11- and 50.

### PREFERRED EMBODIMENTS

It is known that in cold liquids, bubble cloud cavitation is able to drive reactions that normally occur only under extreme conditions. Examples include activation of liquid-solid reactions and synthesis of amorphous and nanophase metals, alloys, metal carbides, and nano-colloids. Another remarkable phenomena occurs during ultrasonic irradiation of liquid-solid slurries: extremely high speed inter-particle collisions. Turbulent flow and shock waves produced by acoustic cavitation can drive metal particles together at sufficiently high velocities to induce melting upon collision. see Abstract Kenneth S. Suslick, Multi-Bubble Sonoluminescence, Department of Chemistry and of Material Science & Engineering University of Illinois at Urbana-Champaign, 601 S. Goodwin Avenue Urbana, Ill. 61801

As a backdrop to our invention, it is largely held to be true that mild forms of magnetic energy, including low to extremely low magnetic energy fields, serve to ameliorate and treat a variety of common human ailments and complaints. It is anecdotally noted that, in our society, “magnet” therapy is used for everything from insoles for shoes, to mattress covers, to jewelry, with proponents of such “magnet therapies” claiming that the use of “magnets” anecdotally and subjectively makes them feel healthier or better, and/or improves some aspect of their everyday life.

In recent years, the medical and related sciences have begun to look at magnets and “magnet therapy” more closely. It is now largely understood that the exposure of living organisms to a magnetic fields of a variety of ranges, dependent upon the intended purpose (from extremely low frequency fields to a high amplitude magnetic fields), can offer a number of distinct health benefits and advantages.

In the last several years it has been shown that magnetic field therapy holds a number of disparate yet significant and important findings related to proper physical health and functioning.

A number of recent studies have been performed with magnetic fields on the brains of rats, to find aids in neuronal medicine. It has been discovered via these techniques that extremely low frequency magnetic fields protect cells from focal brain injury (contusion), with a showing of significantly decreased inflammatory reaction via cascade and neuronal injury following such as focal brain injury in the rats which had been exposed to such a field, as compared to those rats which had not.⁽²⁸⁾Additionally, exposure to an extremely low frequency magnetic field not only spares rat neurons from injury, it has also been shown to save rat cerebellar granule neurons from apoptosis or early cell death in some settings.⁽²⁹⁾And, in the rat brain, the optimization of administered lithium has been shown to be positively effected in the presence of a magnetic field. thus stabilizing the neuronal cells.⁽³⁰⁾

There is also known to be a large number of neural cells, including GABA and serotonin cells, in the human digestive tract or gut. Human gastrointestinal functioning is now thought to be enhanced by exposure to a specific magnetic field in the area of the intestines, in a sense re-training one's body to function more normally following a major event such as abdominal surgery.⁽³¹⁾

Very weak ultra-low magnetic fields of a pulsating nature have been described as raising pain thresholds and providing analgesic benefits on sensory and pain thresholds. ⁽³²⁾Indeed, studies have pointed to the analgesia following exposures to magnetic fields as being facilitated by the alpha-adrenergic system in a dose-dependent fashion, and in fact such magnetic field therapy is thought to be an important and powerful adjunct to pharmacologic treatments of pain.⁽³³⁾

There is some evidence, too, that the influence of static magnetic fields effects mechanosensitive ion channel activity on artificial liposomes, which may lead to an increased possibility of the liposome becoming trapped open on purpose, which may hold promise for persons attempting to lose or maintain weight, as the opening of the liposome is a required step in ordinary weight loss.⁽³⁴⁾

Related to weight loss is the problem of free radical creation in an in-vivo environment as a result of the lipid oxidation which is a necessary step in the weight loss process. There is now beginning to be interest in how a magnetic field may act to help alleviate such problems with free radicals.⁽³⁵⁾In one example, weak electric fields are shown to indirectly effect endogenous signaling pathways involving the calcium channels and adherent and motile neutrophils, NAD(P)H concentration, flavoprotein redox potential, and the production of reactive oxygen species and nitric oxide in vivo, providing the ability to control these facets of some cells functioning by altering the magnetic fields in question.⁽³⁶⁾Blood oxygenation, viscosity, and flow are also shown to be in part modulated by exposure to low frequency magnetic fields.⁽³⁷⁾

Immune protection is perhaps a more important topic than ever with issues such as potential global bio-terrorism, and the decline of effectiveness of antibiotics take root in our society. Magnetic fields have been shown to provide assistance to host immunity at a cellular level. For instance, extremely low frequency magnetic fields positively affect phagocytic activity and also are responsible for enhanced interleukin (1L-1 beta) production in the host cell, which is critical for cell immunity and survival of the host.⁽³⁸⁾

Additionally, it has been noted that magnetic fields also enhance the proliferation activities of lymphocytes, relating this effect to activities related to the calcium ion influx in such treated cells.⁽³⁹⁾

Magnetic fields used in conjunction with newer nano-particles have likewise been paving the way for significant advances in the field of cancer treatment. In one instance, magnetic nanoparticles were conjugated to a monoclonal antibody and then delivered by i.v. to reach the target mouse cancer tissue. Then, alternating magnetic fields were applied to cause heating, thus selectively destroying the cancerous tissues and sparing thr normal tissues.⁽⁴⁰⁾In another case, magnetic doxorubicin nanoparticles were made via supersonic mixer and drying, and then introduced for tumor treatment under a magnetic field to evaluate its heat effect, which was well-tolerated with a steady transfer to the surroundings, thereby providing another potential avenue for cancer treatment.⁽⁴¹⁾

And last but perhaps not least, with beauty as a mainstay of our modern culture, it is of note that two features of skin healing, namely, the quality of tissue repair, and the speed with which such repair can be accomplished, are both positively impacted by magnetic field exposure. Using magnetic fields for traumatized or sensitive areas of skin are now shown to be a promising tool in the healing process.⁽⁴²⁾

### Specification

We lend the following methods and improvements to the nearest non-essential art pieces as above, and have conceptually developed our own novel techniques as specified herein. Not only are our techniques novel, but also our resultant supraparamagnetic particles are novel as well, in that they have not been exposed, described, demonstrated, or explored, either theoretically or actually, in any publication, patent, or patent application, foreign or otherwise, heretofore.

For our invention, it is first necessary to have a completely spherical non-reactive apparatus to serve as the luminescence vessel. Such a vessel may be made of glass, plastic, quartz, ceramic, and the like. Of note on the topic of novelty is that while other inventions may show that various shapes of sonoluminesence vessels such as cylindrical or mono transducer may be inherently insufficient for sustained fusion or for coupling and or compression of atoms or nano particle molecules. Sperical, elliptical shaped vessels or dewars more naturally mimic the natural shape of galaxies and fluid flow analogous to dark and light matter movement in Galaxies as discussed in SGS. In previous art, the shape of vessel used is rather interchangeable, not requiring one shape over another. This interchangeablilty of prior art is not applicable for our invention. Our invention requires a completely spherical vessel. In fact, for our invention, the particles contained within the vessel are required to move in specific spherical fluid flow dynamics as a requirement in order for the compression of the flow of the electrons to occur more efficiently, in a direct and balanced manner, this aforementioned quality only being achieved in a completely spherical vessel. Please see FIG. 1 as contained in this application to crudely show the necessary components and the fluid dynamics in our required spherical vessel. We contend that the fact that our present invention requires a spherical vessel allowing such spherical flow dynamics, further serves to differentiate our work from previous works.

Additionally, we have added the novel concept of fitting the inert spherical vessel with two transducers; one positive transducer at the top of the sphere, and one negative transducer at the bottom of the sphere. A double transducer model has not previously been described in the literature.

Specifically, our transducers are made from standard sizes presently utilized to small nanotechnology various torus-designed structures for vortex wave generation. Examples of possible torus transducers include, but are not limited to, metals, ceramics, glass, and fiberoptics, with metals being our preferred substrate of choice in regards to achieving a supraparamagnetic state with the most ease. Our torus transducers are geometrically shaped like a three-dimensional washer, such as a donut, vortex ring, and also micro to macro parabols as below. Simple tests and analogies show this will show the vortex apex of our items proportionately smaller in radius with the smaller the size of the torus transducer both in circumference and radius of the inner donut hole, as well as other dimensions of the torus transducer. Dynamics or the wave form has puzzled many of the best scientists in this areas. However, analogous tranducer testing is the treatment of cancer in the human body make it clear that a parabol transducer makes a vortex with vaporization of tissue at the apex are much the same way the gas or water is vaporized. One benefit of this analogy is that the gas and tissue destruction remain in the tissue sample or body for some time after word allowing tissue analysis to help with great precision which frequencies and amplitudes resulted in the in damage in specific distances from the transducer See F. L. Lizzi, One line assessment of HIFU beams at http://www.rri-usa.org/biomed/publications/pdf/Lizzi_{—}2004_IEEE_On-line.pdf

We are proposing the use of sets of ceramic transductors such as toruses or diminishing in size parabolic transducers placed on opposing ends of the sphere, with crossover electrical coils, ripples or fibers placed on the surface of both sets of paraboles, such that created are superimposed waves carried a further distance on a primary or secondary wave, such that the energized matter meets in the center of the sphere, for the particles to be acted upon there.

There will be dual transducers facing each other at the poles of the spherical vessel Examples of such would be vaper-doped metal ions onto a substrate in the micro range to generate tall thin vortexes. Most of the tall vortex shapes will result in coupling with each other in the nucleus area via apex in the vessel. Herein is our novel inventive entity. This tall vortex coupling can be most accurately described as a pseudo-mausbauer effect, with a vortex-pinning effect like a screwing of the metal down and compressing of the electrons close to the nucleus so as to increase magnetic field and thus increase the temperature required for supramagnetic and superconducting properties, yet otherwise maintaining virtually every of the compound's original and known properties such as density, atomic weight, and oxidative states.

For the sonoluminesence medium, we propose in addition to water and argon, (or other noble gasses) the use of helium superfluid medium and perhaps alternatives of argon gas or nitrogen gas for the nucleus in lieu of helium. see Abstract of V. Penna Turin Politecnico—Italy The effects of confinement on the quantization of vortex-antivortex pairs (Minimum energy configurations are investigated for a vortex-antivortex pair in a box with a circular boundary. The appropriate algebraic scheme for quantizing vortex dynamics within this confined geometry and the structure of the vortex pair spectrum are discussed at http://ochsenkopf.physik.uni-konstanz.de/˜qfs2001/˜qfs2001_abs/node18.html.). The effect of this medium and or in combination with a standard torus or parabol transducer for making longer vortexes than non superfluid medium. In addition, coupling with the medium and the metals will occur due to cooper pair bonding will allow coupling of the now higher temperature superconducting metals in the vessel for easier coupling and compression adding to longer term higher temperature superconducting status even after removal. Alternately, one could use an oil or other traditional fluid medium in which the metallic particle spheres will remain suspended in the nuclear zone of the vessel which over the short period of time which it will take to yield the finished supraparamagnetic product.

This present invention provides methods for novel radioprotective materials, in the form of nano to micro spheres as compositions with greater supramagnetic field strength and higher temperature super conducting magnetic field features than have been described to date.

In this present invention as mentioned above, metals will be placed into a spherical sonolumenescence vessel as above, in which is contained a gas of argon, nitrogen, or helium, or fluid such as oil or other traditional fluid medium as above. The metallic particle spheres that are introduced as in our invention will remain suspended in the nuclear zone of the vessel which over a short period of time while the current of energy flows, thereafter yielding the finished supraparamagnetic product.

It is important to note that alternately, other forms of energy may be used to accomplish the creation of such a paramagnetic metal product, such as applying a great amount of direct or indirect physical pressure to the metal. For instance, magnetic pressure can be served upon the metals in question such as by using a standard Magnetic Resonance Imaging machine in order to bring about the supraparamagnetic effect of these metals.

Unexpectedly and interestingly, however, is that all of these methods herein of pressurizing the metal will leave the metal grossly intact and essentially unchanged in regards to the chemical's ordinary attributes such as boiling point, melting point, and density. Various other attributes of the metal on an atomic level will show changes, such as a shifting or an alteration of the chemical's electron configuration upon the metal reaching into the supraparamagnetic realm. Thus, in most cases the resulting modified metal particles will largely remain as safe as is the original chemical itself.

Therefore, our supraparamagnetic particles are largely safe for in-vivo delivery or ingestion, including intradermal and intramuscular use, and topical application as a liquid, créme, gel, or transdermal patch. In a variety of situations, these novel particles may be easily useable for preventative and therapeutic purposes in the human body for purposes including but not limited to: blocking the harmful effects of various types of radiation (exposure to which occurs unconsciously in daily life and even in some working environments); potentially reducing the number of abnormally rapid, premature, or irregular cell divisions; maintaining cell fluid stability and integrity; and the targeted treatment of specific disease states in some persons, such as may be determined by the type of illness and the genetic make-up of the host.

These radioprotective materials will largely come from, either used alone or invarious combinations: the anion, cation, and/or neutral atoms or oxides of metals from the group comprising titanium, cobalt, chromium, molybdenum, niobium, yitrium, zirconium, tantalum, sulfur, hafnium, lanthanum, rhenium, sodium, rhodium, magnesium, beryllium, lithium, tellurium, scandium, calcium, ruthenium, tungsten, osmium, iridium, technetium, sulfur, polonium, manganese, carbon, gadotinium, platinum, and vanadium. Additionally, in certain disease conditions or states, it might be necessary in select from additional specific metals including iron, copper, aluminum, boron, selenium, silicon, iodine, gold, zinc, silver, bismuth, and potassium in order to affect the illness at hand. Various such treated materials, including alloy or isotope combinations of both sets of the before mentioned metals, used alone or in conjunction with similarly treated helium coated nanospheres or bathing in an MRI machine, will increase the bioavailability and therefore increase the therapeutic benefits of other medications or supplements. Moreover the metal Platnium has properties known to absorb or neutralize oxygen free radicals. Our novel metal particles can act to selectively target a specific organ system or function of the body by being integrated into human or bovine glandular supplements and extracts, as well as a variety of now common-place oral and liquid dietary supplements such as chondroitin, melatonin, glucosamine sulfate, biotin, methylsulphonylmethane, taurine, etc.

These same stabilizing and radioprotective properties can be useful for widespread shielding matters, using the treated materials inside of and outside of the body in order to provide partial or more complete screening for the body itself. Such applications may include integration into various building compounds, use of the material(s) in surgical applications of a therapeutic and/or cosmetic nature, wearing the paramagnetic metal(s) within an amulet or other personal device, integrating the metal(s) into hair, cosmetic, and body care products and/or tattooing inks, and also applying the metal(s) by spraying it onto or incorporating it into woven fibers or materials such as clothing or blankets.

### DRAWINGS

FIG. 1 is a Side Cut-Away View of the Spherical Sonoluminesence Vessel which acts as a Particle Magnetizer which holds the Dual Transducers at either end.

A shows the positively charged vortex which is generated by the transducer B.

B is the outer shell of the spherical dewar medium.

C is the outer containment vessel wall of the dewar.

D is the spherical dewar medium.

E is the center point fusion area.

F is the particle inlet and outlet area into the dewar.

G is the second transducer.

H is the negative vortex generated by the transducer G.

I, J and K demonstrate suspended nano-particles.

FIG. 2 is a Side Cut-Away View of a Blow-Up of The Center Point Fusion Area (which is Area E of FIG. 1 above). FIG. 2 shows the initial phase of the collision of the nano-particles located at the Vortex Apexes of the colliding vortexes, showing the North Pole and South Pole of the nano-particles and showing the confinement or coupling of the particles which occurs in this center point fusion area.

A shows the wave of the positively charged vortex

B shows the leading edge of the apex of the incoming vortex wave interfacing or coupling or pinning like a Phillips screwdriver with the electron shell of the nanoatom subject suspended in the medium of the dewar acting to bring the atoms of the subject atom closer together., compressing the atom, maximally twisting the electron shell of the atom, in order to bring the entity into a higher energy state.

C shows the wave of the negatively charged vortex

D shows the leading edge of the apex of the incoming vortex wave interfacing or coupling or pinning like a Phillips screwdriver with the electron shell of the nanoatom subject which is suspended in the medium of the dewar actinso as to bring the atoms of the subject atom closer together, compressing the atom, maximally twisting the electron shell of the atom in order to bring the entity into a higher energy state.

E shows that this field is within a range of Nanometers in size, from 1 Nanometer in size range to 200 Nanometers in size range, depending upon the size of the atom or atoms used.

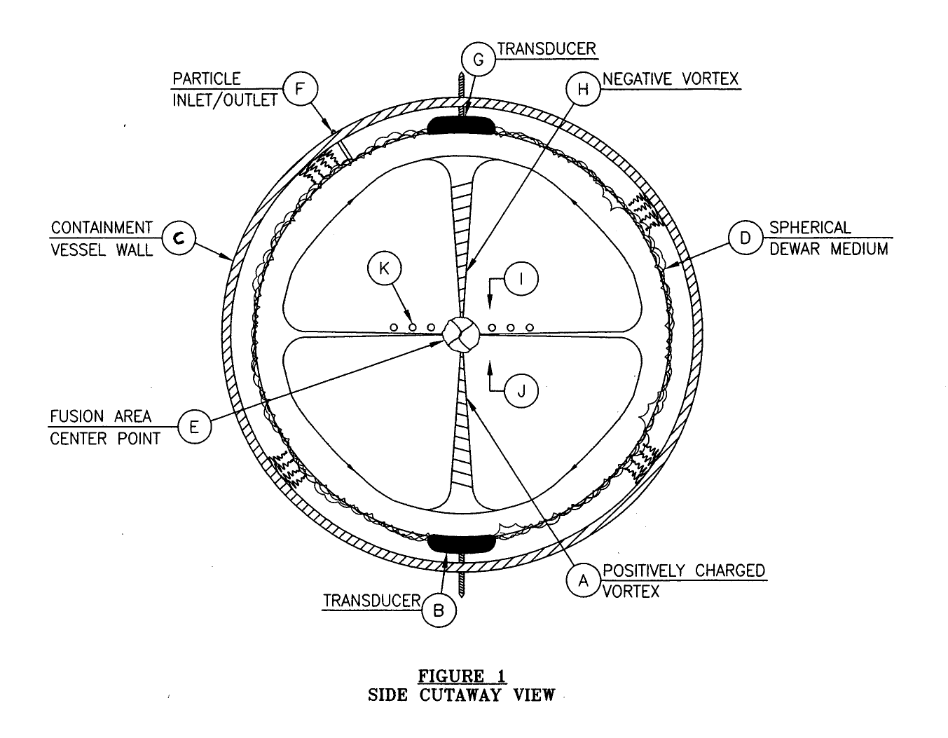

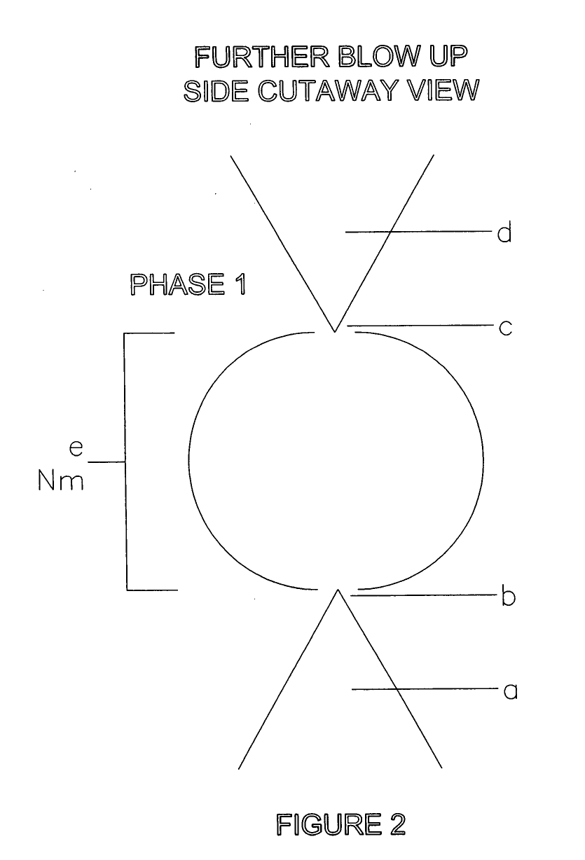

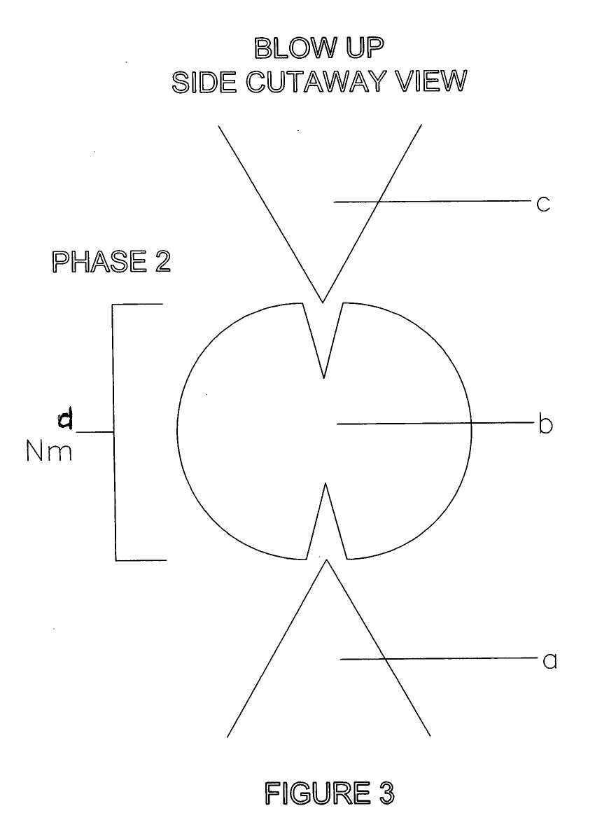

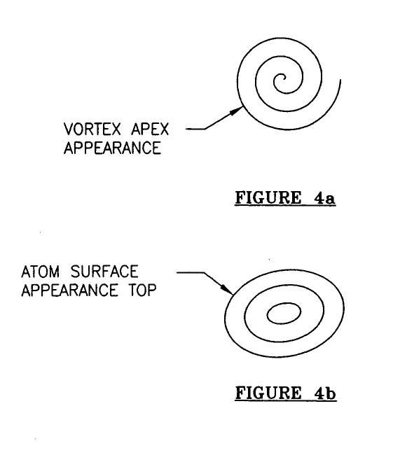

FIG. 3 is a Further Side Cut-Away View of a Blow-Up of The Center Point Fusion Area (which is Area E of FIG. 1 above) FIG. 3 is a time-lapsed drawing, showing the pinning down or twisting down process of the atoms as it begins to occur with additional compression of the atom, and additional twisting of the electron shell of the atom, to bring about a higher energy state, increasing the magnetic field or crystallization of the nano-particles, and bringing about a coupling effect between atoms.

A shows the wave of the positively charged vortex

B shows the leading edge of the apex of the incoming vortex wave continuing to interface or couple or pin like a Phillips screwdriver with the electron shell of the nanoatom subject suspended in the medium of the dewar continuing to act to bring the atoms of the subject atom closer together, continuing to compress the atom, even more maximally twisting the electron shell of the atom, in order to bring the entity into an even higher energy state.

C shows the wave of the negatively charged vortex

D shows that this field is within a range of Nanometers in size, from 1 Nanometer in size range to 200 Nanometers in size range, depending upon the size of the atom or atoms used.

FIG. 4a is the aerial view of the vortex apex.

FIG. 4b is the aerial view of the topography of the nano-particles, which depicts how they couple, confine, or interface each with a vortex apex . . . .

### REFERENCES

1. R. P. Taleyarkhan, C. D. West, J. S. Cho, R. T. Lahey, Jr. R. Nigmatulin, and R. C. Block, Evidence for Nuclear Emissions During Acoustic Cavitation, Science 295, 1868 (2002). (available online)
2. D. Shapira, M. J. Saltmarsh. Comments on Reported Nuclear Emissions during Acoustic Cavitation, 1 Mar. 2002. (available online (PDF))
3. R. P. Taleyarkhan, R. C. Block, C. D. West and R. T. Lahey Jr., “Comments on the Shapira and Saltmarsh Report” 2 Mar. 2002. (available online (PDF))
4. F. Becchetti, Evidence for Nuclear Reactions in Imploding Bubbles, Science 295, 1850 (2002).
5. D. Kennedy, To Publish or Not to Publish, Science 295, 1793 (2002).
6. New Energy Times Newsletter 18 (Bubble Fusion Discoverer Taleyarkhan Strikes Back)
7. D. Shapira, M. J. Saltmarsh. Nuclear Fusion in Collapsing Bubbles Is it There? An Attempt to Repeat the Observation of Nuclear Emissions from Sonoluminescence, Phys. Rev. Lett. 89, 104302 (2002).
8. A. Galonsky. Tabletop Fusion Revisited Science 297, 1645 (2002).
9. C. Murry. An Experiment to Save the World BBC Horizons, aired February 2005. (BBC News story) (Transcript of the program)
10. Bourgeois, Theresa. “Researchers Report Bubble Fusion Results Replicated: Physical Review E publishes paper on fusion experiment conducted with upgraded measurement system”, RPI News & Information, Mar. 2, 2004. Retrieved on 2006-12-05.
11. R. P. Taleyarkhan et al., Additional Evidence of Nuclear Emissions During Acoustic Cavitation, Physical Review E 69, 036109, 22 Mar. 2004 (abstract available online)
12. Venere, Emil. “Purdue findings support earlier nuclear fusion experiments”, Jul. 12, 2005. Retrieved on 2006-12-05.
13. Y. Xu, A. Butt Confirmatory Experiments for Nuclear Emissions During Acoustic Cavitation Nuc. Eng. Des. 235, 1317 (2005).
14. Peplow, Mark (2006-01-10). “Desktop fusion is back on the table”. Nature. DOI:10.1038/news060109-5. Retrieved on 2006-12-05.
15. R. P. Taleyarkhan, C. D. West, R. T. Lahey, Jr., R. I. Nigmatulin, R. C. Block, and Y. Xu. Nuclear Emissions During Self-Nucleated Acoustic Cavitation, Phys. Rev. Lett. 96, 034301 (2006). (abstract available online)
16. Jan. 31, 2006, Sciencedaily: Using Sound Waves To Induce Nuclear Fusion With No External Neutron Source Quote: “ . . . The experiment was specifically designed to address a fundamental research question, not to make a device that would be capable of producing energy, Block says . . . To verify the presence of fusion, the researchers used three independent neutron detectors and one gamma ray detector. All four detectors produced the same results: a statistically significant increase in the amount of nuclear emissions due to sonofusion when compared to background levels . . . ”
17. (Received 19 Sep. 2005; published 27 Jan. 2006), Physical Review Letters: Nuclear Emissions During Self-Nucleated Acoustic Cavitation Quote: “ . . . Statistically significant nuclear emissions were observed for deuterated benzene and acetone mixtures but not for heavy water. The measured neutron energy was <=2.45 MeV, which is indicative of deuterium-deuterium (D-D) fusion. Neutron emission rates were in the range ˜5×10³ n/s to ˜10⁴ n/s and followed the inverse law dependence with distance . . . ”
18. (2006-03-08) “Bubble fusion: silencing the hype”. Nature. DOI:10.1038/news060306-1. Retrieved on 2006-12-05.
19. Naranjo, Brian (Oct. 3, 2006). “Comment on “Nuclear Emissions During Self-Nucleated Acoustic Cavitation””. Phys. Rev. Lett. 97 (149403). DOI:10.1103/PhysRevLett.97.149403.
20. Taleyarkhan, R. P.; R. C. Block, R. T. Lahey, Jr., R. I. Nigmatulin, and Y. Xu (Oct. 3, 2006). “Taleyarkhan et al. Reply:”. Phys. Rev. Lett. 97 (149404). DOI:10.1103/PhysRevLett.97.149404.
21. Eugenie Samuel Reich (2006-03-09). “Evidence for bubble fusion called into question”. Nature 440 (132). DOI:10.1038/440132b. Retrieved on 2006-12-05.
22. Nature on the Attack. Includes links to correspondence and to Nature article.
23. Code of Ethics Society of Professional Journalists
24. Science, Journalism and Nature, New Energy Times article.
25. Bubble Fusion Confirmed by LETU Research”, Le Tourneau University News, 2006-11-17. Retrieved on 2006-12-05.
26. ANS 2006 Winter Meeting & Nuclear Technology Expo Official Program (PDF) (Nov. 12-16, 2006). Retrieved on 2006-12-06.
27. Forringer, Edward R.; David Robbins, Jonathan Martin (November 2006). “Confirmation of Neutron Production During Self-Nucleated Acoustic Cavitation”. Transactions of the American Nuclear Society.
28. Zhao L; Zhao D M; Wei J H; Wang Y Q; Huang Z M; Effect of extremely low frequency magnetic field on the focal brain injury in rats. Space Med Med Eng (Beijing). 2003: 16(1):75-6 (ISSN: 1002-0837)
29. Oda, T; Koike T; Magnetic field exposure saves rat cerebellar granule neurons from apoptosis in vitro. Neurosci Lett; 2004; 365(2):83-6 (ISSN: 0304-3940)
30. McKay B E; Persinger M A; Lithium ion “cyclotron resonance” magnetic fields decrease seizure onset times in lithium-pilocarpine seized rats. Int Neurosci. 2004; 114(8):1035-45 (ISSN: 0020-7454)
31. Bradshaw L A; Myers A; Wikswo J P; Richards W O; A spatio-temperol dipole simulation of gastrointestinal magnetic fields IEEE Trans Biomed Eng. 2003: 50(7):836-47 (ISSN: 0018-9294)
32. Shupak N M; Prato F S; Thomas A W; Human exposure to a specific pulsed magnetic field: effects on thermal sensory and pain thresholds. Neurosci Lett. 204; 363(2):157-62 (ISSN: 0304-3940)
33. Martin L J; Persinger M A; Thermal analgesia induced by a 30-min exposure to 1 microT burst-firing magnetic fields is strongly enhanced in a dose-dependent manner by the alpha2 agonist clonidine in rats. Neurosci Lett. 2004: 366(2):226-9 (ISSN: 0304-3940)
34. Hughes S; El Hal A J; Dobson J; Martinac B; The influence of static magnetic fields on mechanosensitive ion channel activity in artificial liposomes; Eur Biophys J. 2005; 34(5):461-8 (ISSN: 0175-7571)
35. Deng Y; He G; Petrakov S; Kuppusamy P; Zweler J L; Fast EPR inaging at 300 MHz using spinning magnetic field gradients. J Magn Reson. 2004: 168(2):220-7 (ISSN: 1090-7807)
36. Rosenspire A J; Kindzelski A L; Simon B J; Petty H R; Real-time control of neutrophil metabolism by very weak ultra-low frequency pulsed magnetic fields. Biophys J. 2005: 88(5):3334-47 (ISSN: 0006-3495)
37. Yamamoto T; Nagayama Y; Tamura M, A blood-oxygenation-dependent increase in blood viscosity due to a static magnetic field. Phys Med Biol. 2004: 49(14):3267-77 (ISSN: 0033-9155)
38. Frahm J; Lantow M; Lupke M; Weiss DG; Simk M; Alteration in cellular functions in mouse macrophages after exposure to 50 Hz magnetic fields. J Cell Biochem. 2006: 99(1):168-77 (ISSN: 0730-2312)
39. Deng Y; He G; Petrakov S; Kuppusamy P; Zweler JL; Fast EPR inaging at 300 MHz using spinning magnetic field gradients. J Magn Reson. 2004: 168(2):220-7 (ISSN: 1090-7807)
40. Ivkov R; DeNardo S J; Daum W; Foreman A R; Goldstein R C; Nemkov V S; DeNardo G L; Application of high amplitude alternating magnetic fields for heat induction of nanoparticles localized in cancer. Clin Cancer Res. 2005: 11(19 Pt 2):7093s-7103s (ISSN: 1709-0433)
41. Shi F; Wu M; Zhang M; A study on the preparing of magnetic doxorubicin nanoparticles and its heat effect under a magnetic field. Sheng Wu Yi Xue Gong Cheng Xue Za Zhi, 2004; 20(3):463-5 (ISSN: 1001-5515).
42. Pate K; Benghuzzi H; Tucci M; Puckett A; Cason Z; Morphologic evaluation of MRC-5 fibroblasts after stimulation with static magnetic field and pulsating electromagnetic field. Biomed Sci Instrum. 2003; 39:460-5 (ISSN: 0067-8856)

### EXTERNAL LINKS

“Bubble Power”, Richard T. Lahey Jr., Rusi P. Taleyarkhan & Robert I.
Nigmatulin, IEEE Spectrum Magazine, May 2005—Readable, quantitative, illustrated article
“Bubble Fusion Research Under Scrutiny”, IEEE Spectrum, May 2006, follow-up on May 2005 article
R. T. Lahey Jr, R. P. Taleyarkhan, and R. I. Nigmatulin, Sonofusion—Fact or iction? (PDF format)
Sonofusion—Directory page at FreeEnergyNews.com, a newsletter/website which promotes zero cost energy schemes
“Possible Sound-Induced Nuclear Fusion Posited” Rensselaer Polytechnic Institute Press Release, Mar. 5, 2002
“Fusion controversy rekindled” BBC News, Mar. 5, 2002
“Fusion experiment disappoints” BBC News, Jul. 2, 2002
“Evidence bubbles over to support tabletop nuclear fusion device” Mar. 2, 2004
“Sound waves size up sonoluminescence”. PhysicsWeb. Feb. 5, 2002
“Researchers Report Bubble Fusion Results Replicated” Rensselaer Polytechnic Institute Press Release, Mar. 2, 2004
Harnessing bubbles to trigger nuclear fusion—22 Jan. 2005, Justin Mullins, New Scientist Magazine Issue 2483 (subscription required)
Purdue findings support earlier nuclear fusion experiments—New positive bubble/sonofusion findings were detailed in a peer-reviewed paper appearing in the May issue of the journal Nuclear Engineering and Design. (Jul. 12, 2005)
Bubble Fusion takes next hurdle—The potential for cavitation to induce nuclear fusion lets physicists think in new directions of energy production. (Jul. 18, 2005)
“Desktop fusion is back on the table; Physicist claims to have definitive data. but can they be replicated?”, news nature.com, Jan. 10, 2006 (subscription required)
“Sonofusion Experiment Produces Results Without External Neutron Source” PhysOrg.com Jan. 27, 2006
“Using Sound Waves To Induce Nuclear Fusion With No External Neutron Source” (sciencedaily.com, Jan. 31, 2006)
“Bubble fusion: silencing the hype”, Nature online, Mar. 8, 2006—Nature reveals serious doubts over claims for fusion in collapsing bubbles (subscription required)
What's New. Mar. 10, 2006-failed replications
“Bubble-fusion group suffer setback; Team admits a mix-up with one of their neutron detectors”, May 10, 2006, news@nature.com (subscription required)
“Purdue Bubble Wraps Sonofusion Inquiry Results”, Jun. 21, 2006 Photonics.com
“Chain Reaction” Movie, August 1996
“New Energy Times”
“Introduction to Sonofusion” Roger Stringham, October 2006

1. A method for making a radioprotective and otherwise beneficial material by the technique of applying physical and/or magnetic, and/or sound and/or light pressure such as via sonolumenesce in an inert spherical vessel with a transducer with a positive charge at one end and a transducer with a negative charge on the opposite end, with the suspended item being either helium and one or more of the anions, cat ions, oxides, or isotopes of the metals, from the group comprising titanium, cobalt, chromium, molybdenum, niobium, yitrium, zirconium, tantalum, sulfur, hafnium, lanthanum, rhenium, sodium, rhodium, magnesium, beryllium, lithium, tellurium, scandium, calcium, ruthenium, tungsten, osmium, iridium, technetium, platinum, manganese, vanadium, iron, copper, sulfur, aluminum, boron, selenium, polonium, silicon, carbon, iodine, bismuth, zinc, and potassium, or a liquid based medium and one of the anions, cations, oxides, or isotopes of the metals, from the group comprising titanium, cobalt, chromium, molybdenum, niobium, yitrium, zirconium, tantalum, sulfur, hafnium, lanthanum, rhenium, sodium, rhodium, magnesium, beryllium, lithium, tellurium, scandium, calcium, ruthenium, tungsten, osmium, iridium, technetium, platinum, manganese, vanadium, iron, copper, sulfur, aluminum, boron, carbon, selenium, polonium, silicon, iodine, bismuth, zinc, and potassium.

2. A material as that made as in claims #1, whereby the supraconducting material is then bathed in superfluid helium in order to further activate or maximize the higher-temperature superconducting properties of the material.

3. A material as that made via any one of claims #1 or #2, whereby the supraconducting material is then bathed in, and/or periodically refreshed in, a mild to moderate superconducting magnetic field such as that of an MRI machine, or by superimpostion to compound the supraconducting magnetic fields in order to enhance, lengthen, or maintain the supraconducting properties of the material.

4. A material such as that made in claim #1, #2, or #3, which can be taken orally, applied via skin patch, injected, or dripped with an IV, where the resultant entity stabilizes cells in vivo, thus reducing cell turnover, and maximizing the longevity of the cell, to make possible gains or increases in human, animal, and insect longevity.

5. A material such as that made in claim #1, #2, or #3, which can be taken orally, applied via skin patch, injected, or dripped with an IV, where the entity stabilizes healthy cells in vivo, thus reducing irregularities and certain abnormalities in cell division, and therefore diminishing the risk of the development of certain types of cancers later in life.

6. A material such as that made in claim #1, #2, or #3, which can be taken orally, applied via skin patch, injected, or dripped with an IV, where the resultant entity can be coated with biological or chemotherapeutic agents before introduction into the body, in order to target both free cancer cells, and also specific cancer cells in specific organs of the body, with biodelivery of the item directly to the diseased organ(s) via the optional use of oral or intramuscular bovine or human glandular substances.

7. A material such as that made in claim #1, #2, or #3, which can be taken orally, applied via skin patch, injected, or dripped with an IV, where the resultant entity can be coated with a stabilized form of ozone, alone or in addition to one or more biological or chemical agents, in order to target both cancer cells, and/or cells of one or more anaerobic disease-causing entities, also cancer cells in various organs of the body, with biodelivery of the entity directly to the diseased organ(s) via the optional use of bovine or human glandular substances.

8. A material such as that made in claim #1, #2, or #3, which can be taken orally, applied via skin patch, injected, or dripped with an IV, where the resultant entity can be coated with biological and/or antimicrobial agents in order to target, by the use of a genetic marker or other system, an infectious agent such as a bacteria, virus, fungus, or parasite, thus directing the antimicrobial agent thereto.

9. A material such as that made in claim #1, #2, or #3, where the resultant entity can be used to coat over or to be applied directly or indirectly to the skin, hair, and nails of an individual so as to decrease the detrimental long-term effects of radiation, particularly for persons who engage in frequent air travel.

10. A material such as that made in claim #1, #2, or #3, where the resultant entity can be applied or sprayed onto a skin wound to facilitate healing, or applied topically to or injected into, an area of skin wrinkles, imperfections, or scars, alone or with optional collagen, vitamins, and/or a fat-based derivative, in order to slow the skin damage process and facilitate healing in part by providing radioprotective material where it is crucially needed to create or to maintain a youthful appearance.

11. A material such as that made in claim #1, #2, or #3, where the resultant entity can be applied topically or injected, optionally with collagen, vitamins, and/or a fat-based derivative, onto and/or around the lip area, thus providing radioprotective material where it is crucially needed around the mouth and lip area, so as to help to create or to maintain a more youthful appearance.

12. A material such as that made in claim #1, #2, or #3, where the resultant radioprotective entity can be applied to, sprayed onto, or incorporated into fabrics and inks of various kinds, for use in tattooing and in making clothing, blankets, drapes, and the like, in order to provide a modicum of radioprotection as part of everyday life.

13. A material such as that made in claim #1, #2, or #3, where the resultant radioprotective entity can be applied to, sprayed onto, or incorporated into cement, woods, and other building substances for use in the housing market, especially for those persons with illnesses which could be exacerbated by unnecessary radiation exposure.

14. A material such as that made in claim #1, #2, or #3, where the resultant radioprotective entity can be placed into, applied to, sprayed onto, or incorporated into jewelry, watches, and other such personal items.

15. A material such as that made in claim #1, #2, or #3, where the resultant entity may serve as a component of permanent or semi-permanent therapeutic or cosmetic medical devices which are implantable into the body.

16. A material such as that made in claim #1, #2, or #3, where the resulting entity helps minimize the damage caused by free radical release during weight loss, thereby facilitating weight loss and weight maintenance.

17. A material such as that made in claim #1, #2, or #3, where the resulting entity has commercial and/or industrial benefits due to its supramagnetic properties.
